(19) United States
(12) Patent Application Publication
(10) Pub. No.: US 2017/0296148 A1
(43) Pub. Date: Oct. 19, 2017

(54) MEDICAL IMAGING SYSTEM AND METHOD

(71) Applicant: Signostics Limited, Clovelly Park (AU)

(72) Inventors: Andrew John Niemiec, Seaton (AU); Essa El-Aklouk, Hillcrest (AU)

(21) Appl. No.: 15/485,935

(22) Filed: Apr. 12, 2017

(30) Foreign Application Priority Data
Apr. 15, 2016 (AU) 2016901418

(51) Int. Cl. A61B 8/08; A61B 8/00

(52) U.S. Cl. CPC A61B 8/5223; A61B 8/461; A61B 8/0833; A61B 8/4444

(57) ABSTRACT

An ultrasound probe user's capture of image frames of a patient's organ is assisted by capturing one or more image frames of the ultrasound probe's a field of view; processing the captured image frames to detect a predetermined anatomical feature of the patient having a known positional relationship with the organ; and directing the user to adjust the ultrasound probe so as to locate the organ within its field of view, based on the known positional relationship.

### TECHNICAL FIELD

The present invention relates to a method and system for imaging an organ, such as a bladder. In a typical application, embodiments of the present invention may be used to produce images suitable for obtaining a volume estimation of an organ.

### BACKGROUND

Non-invasive bladder volume measurement techniques with ultrasound sonography have been described in the art. In principle, ultrasound scanning measures distance based on echo travel time. Early echo techniques used a single ultrasound transducer and echo presentation was recorded as echo amplitude versus depth. A method for determining bladder volume to determine residual urine volume based on distance measurement to the dorsal posterior bladder wall was described in the 1960's. The method was not adjusted to specific, filling dependent, measuring configurations.

A relation between the difference in echo travel time between echoes from the posterior and anterior bladder wall and the independently measured bladder volume was recognised. Volume measurement methods based on this observation have been described. These methods are exclusively based on bladder depth measurement. However, since the bladder changes in shape when filling, a single distance measurement is unlikely to provide sufficiently precision for estimation of the entire bladder volume where no filling dependent measurement configuration is used.

Diagnostic ultrasound is today well known for real-time cross-sectional imaging of human organs. For cross-sectional imaging an acoustic beam is swept electronically or mechanically through a cross section to be imaged. Echoes are presented as intensity modulated dots on a display, to provide the well-known ultrasound sector scan display. Bladder volume may be calculated based on bladder contours obtained in two orthogonal planes with a geometric assumption of bladder shape. For three-dimensional or volumetric sonography, the acoustic beam is swept through the entire organ.

HAKENBERG ET AL: “THE ESTIMATION OF BLADDER VOLUME BY SONOCYSTOGRAPHY”, J Urol, VoI 130, pp 249-251, reported a simple method for calculating bladder volume based on measuring the diameters of the bladder from a cross sectional image taken along the midline sagittal bladder plane only. These diameters give the height and depth of the bladder at the scan plane. The bladder volume is then estimated as the product of the height and depth multiplied by an empirically derived constant.

The above approach led to a method used in the current art involving performing one or more two-dimensional diagnostic ultrasound ‘B’ scans to produce images of one or more cross sections through the structure whose volume is of interest, such as the bladder, and then to make several standard reference measurements of that imaged structure which are then inserted into a formula to estimate the cross sectional area or volume as required. For a bladder, transverse and a longitudinal (sagittal) scans are recorded and the height and width of the transverse image and the depth of the longitudinal one are manually measured, then multiplied together to produce a measure of the volume. A scaling constant is usually also included within the calculation which then crudely models the volume of an oblate ellipsoid.

Unfortunately, such a crude model is likely to have extremely high inaccuracies. As described above, since the bladder varies greatly in shape, an individual's bladder shape will vary according to the degree of filling. Furthermore, between individuals, the shape of a bladder will vary depending on a number of factors, which may change the actual bladder shape of the apparent shape as shown by an ultrasound scan. These factors may include, for example, the presence or absence of a uterus, and the presence or absence of a prostate. Pathology of the bladder, such as, haematoma, or of the surrounding organs, may also distort the bladder, and thus also affect the bladder shape.

One ultrasound apparatus for determining the bladder volume is described in U.S. Pat. No. 4,926,871 (US '871) to Dipankar Ganguly et al. US '871 discloses a scan head (referred to as a sparse linear array) with transducers mounted at predetermined angles such that the acoustic “beams” emitted by the transducer tend to a common point. The volume is calculated according to a geometric model. An apparatus is described for automatic calculation of bladder volume from ultrasound measurements in two orthogonal planes. The device is complex, including a stepper motor for deflecting the acoustic “beams”. It requires a skilled operator to manipulate the scan head in a particular way to obtain the ultrasound measurements.

Volume measurement based on ultrasound sampling of the bladder with a hand guided transducer mounted in a pantograph has been described by Kruczkowski et al: “A non-invasive ultrasonic system to determine residual bladder volume”, IEEE Eng in Medicine & Biology Soc 10TH Ann Conf, pp 1623-1624. The sampling covers the entire bladder, follows a given pattern and is not limited to a single or two cross sections of the bladder. The acquisition procedure is time consuming and thus does not provide instantaneous volume measurement results.

Apparatus exist in the prior art whereby the transducer, and thus the beam, are mechanically swept over the volume of the bladder. In one such apparatus, bladder volume is measured by interrogating a three-dimensional region containing the bladder and then performing image detection on the ultrasound signals returned from the region insonated. The three dimensional scan is achieved by performing twelve planar scans rotated by mechanically sweeping a transducer. The device is thus mechanically complex.

Ganguly et al in U.S. Pat. No. 5,964,710 entitled “System for estimating bladder volume” describes a method for determining bladder volume based on bladder wall contour detection from ultrasound data acquired in a plurality of planes which subdivide the bladder. In each single plane of the plurality of planes N transducers are positioned on a line to produce N ultrasound beams to measure at N positions the distance from front to back wall in the selected plan. From this the surface is derived. This procedure is repeated in the other planes as well. The volume is calculated from the weighted sum of the plurality of planes. In Ganguly's method the entire perimeter of the bladder is echographically sampled in three-dimensions.

Other apparatus exist in the prior art, where although ultrasound techniques are used to measure bladder volume, no ultrasound image is produced. An advantage of such a system is the user does not need clinical training in ultrasound physics to perform a bladder volume measurement, enabling the use of such apparatus by non-ultrasound trained clinicians such as nurses or untrained physicians. One such device is described in McMorrow et al in U.S. Pat. No. 8,167,803 entitled “System and method for bladder detection using harmonic imaging”. Such systems operate when a filled bladder is in view of the ultrasound system. However they are unable to address acquisition errors in the same way a trained human operator is able. For example, if a user places the ultrasound probe in a region where the bladder was partially outside the field of view, prior art as described in McMorrow et al in U.S. Pat. No. 6,884,217 entitled “System for aiming ultrasonic bladder instruments” instructs how to assist the user to re-aim the instrument. However, the system disclosed by McMorrow is only effective if the bladder has urine in the field of view of the system. In other words, where the urine is outside the field of view, the system is ineffective. For example, if the system described in the prior art are aimed at the bowel, and the full bladder is obstructed by bowel, the systems will return a valid reading of 0 ml, even in circumstances where the bladder is full, thus providing a significant measurement error

There exists a need for a method and system for measuring organ size or volume which provides improves accuracy.

### SUMMARY

According to an exemplary version of the invention, there is provided a system for guiding a user to aim an ultrasound probe to capture one or more image frames of an organ of a patient, the system including:

an image processor for capturing one or more image frames of a field of view of the ultrasound probe;

detecting means for processing the one or more captured image frames to detect the presence of a predetermined anatomical feature of the patient having a known positional relationship with the organ; and

guiding means for directing the user to adjust the aim of the ultrasound probe so as to locate the organ within the field of view of the ultrasound probe based on the known positional relationship.

The system may allow a user to accurately locate the organ within the field of view of the ultrasound probe even in circumstances where no part of the organ is in an initial field of view.

Another exemplary version of the invention provides a method of guiding a user to aim an ultrasound probe to capture one or more image frames of an organ of a patient, the method including:

capturing one or more image frames of a field of view of the ultrasound probe;

processing the one or more captured image frames to detect the presence of a predetermined anatomical feature of the patient having a known positional relationship with the organ; and

directing the user to adjust the aim and/or position of the ultrasound probe so as to locate the organ within the field of view of the ultrasound probe based on the known positional relationship.

Another exemplary version of the invention provides a method for determining the volume of urine in a patient's bladder. However, although the invention relates to determining the volume of urine in a bladder, it will be appreciated that embodiments may be applied to determine the volume of any fluid filled bodily structure using ultrasound technology. One embodiment may involve a method for determining the volume of urine in a patient's bladder including the steps of:

operating an ultrasound probe to capture a plurality of ultrasound image frames;

processing the plurality of captured image frames to detect an estimated location of the pubic bone;

using the estimated location of the pubic bone to estimate the location of the bladder;

guiding the user to adjust the placement of the ultrasound probe according to the estimated location of the bladder so as to obtain a plurality of image frames including the bladder; and

processing the plurality of image frames of the bladder to determine the volume of urine.

Detecting an estimated location of the pubic bone may provide a “real-time” landmark which may assist a user to locate the ultrasound probe in a suitable location to accurately image the bladder, even in the event of an empty bladder, and provide additional positional feedback to the user. For example, in the absence of a detected bladder volume, an embodiment may use the estimated location of the pubic bone to provide guidance to the user on probe position.

Preferably, processing the plurality of captured image frames of the bladder to determine the volume of urine includes estimating the location of the perimeter of the bladder, and the cross sectional area of the bladder.

An ultrasound probe according to an embodiment preferably includes an orientation sensor, such as, a gyroscope. In use, a sagittal scan of the bladder may be taken, preferably across the widest portion of the bladder. When the bladder is located within the field of view of the ultrasound probe, the probe unit is then maintained in this position, while the probe unit is rocked to provide a series of 2D image slices (that is, the image frames) through the bladder. Data from the orientation sensor may then be used to track the relative positions of these slices.

In an embodiment, the location of the perimeter and the cross sectional area of the bladder may be determined by applying suitable processing techniques, such as by processing the 2D image slices using methods such as those described below. In the case of the bladder, the perimeter is an area which is fluid filled and hence anechoic. In this respect, although the below description relates to a bladder, it will be appreciated that embodiments may be used to estimate the location of the perimeter of an organ. The volume of the bladder may also be determined from the 2D image slices by applying numerical integration techniques.

The varying quality of the ultrasound images, including image noise adds to the difficulty of establishing the perimeter of an anatomical structure, such as an organ. Accordingly in some embodiments, the nature of the response of tissue to ultrasound is accounted for during image processing. In this respect, in a preferred embodiment, raw radio frequency (RF) scanlines are processed to determine the bladder perimeter. One advantage of such approach is that raw RF scanlines include information which may be removed after the scanlines are processed for visual display. In another embodiment, RF data associated with the RF scanlines may be enveloped and down-sampled to reduce storage and processing power. Although it is preferred that raw radio frequency (RF) scanlines be used, in other embodiments, it is envisaged that algorithms could be applied on a final processed image.

When obtaining an ultrasound scan of a bladder, a typical scanline passing through the bladder will first pass through echoic tissue, then pass through the anechoic bladder, and then pass through echoic tissue again. In an embodiment, a weighted ratio between a standard deviation of the raw RF scanline data within the echoic region and an anechoic region is used to provide a marker for a scanline crossing a wall of the bladder anteriorly and posteriorly.

Some embodiments may further involve applying suitable post processing techniques to improve the accuracy of detecting the perimeter of the bladder of varies sizes and shapes. As will be described in more detail below, suitable post processing techniques may involve one or more of:
rejecting image frames with low mean likelihood;
rejecting image frames with a low number of detected bladder scanlines;
rejecting image frames having a large bladder area and/or centroid change compared to surrounding frames;
rejecting image frames due to large bladder centroid change compared to surrounding valid bladder frames;
interpolating small gaps in an outline of the bladder perimeter; adjusting small jumps in the outline of the bladder perimeter;
detecting very large jumps in outline of the bladder perimeter and keeping regions of higher likelihood;
modifying other outline anomalies using detected contours in surrounding valid frames; and
applying shape constraints on a detected outline of the bladder perimeter and rejecting offending scanlines; and finally smoothing the outline.

In an embodiment, the location and/or shape of a side wall of the bladder may be estimated by applying a difference equation edge detector on a window in close proximity to a first detected bladder line for a first side wall (for example, a right side wall) and in close proximity to the last detected scanline for an opposite side wall (for example, a left side wall) of the bladder. A window may then be used to bound an extent to which a side wall can deviate from the detected bladder posterior wall, to account for a side wall typically having a much weaker outline and may be attenuated by gas/mirror artefacts on one side, and bone artefacts on the other.

Another aspect of the invention provides a system for determining the volume of urine in a patients bladder including:

an ultrasound probe for capturing a plurality of ultrasound image frames;

a memory storing a set of program instructions;

one or more processors programmed with the set of program instructions for execution to cause the one or more processors to:

process the plurality of captured image frames to detect an estimated location of the pubic bone;

use the estimated location of the pubic bone to estimate the location of the bladder;

provide output information guiding the user to adjust the placement of the ultrasound probe according to the estimated location of the bladder so as to obtain a plurality of image frames including the bladder; and

process the plurality of image frames of the bladder to determine the volume of urine in the bladder.

Another aspect of the invention provides a method for determining the volume of urine in a patients bladder including

operating an ultrasound probe to capture a plurality of ultrasound image frames including an image of the bladder;

processing each of the plurality of captured image frames to:

determine the location of the bladder's anterior and posterior walls using an estimated location of the pubic bone;

process scanlines including features of the anterior and posterior wall to select a respective range of scanlines proximal to end points of the anterior and posterior wall;

apply an edge detection filter to each respective range to detect left and right side walls of the bladder based on intensity transitions across the extent of each respective range; and

determine a volume slice using the detected walls of the bladder;

determining the volume of urine in the bladder as the summation of each volume slice.

### BRIEF DESCRIPTION OF DRAWINGS

Embodiments of the present invention will be discussed with reference to the accompanying drawings wherein:

FIG. 1 is a is a schematic diagram of an ultrasound scan system including an embodiment of the invention;

FIG. 2 is a functional block diagram of an ultrasound system according to an embodiment;

FIG. 3 is a functional block diagram of a digital signal processing (DSP) operation suitable for use with the ultrasound system of FIG. 2;

FIG. 4 is a functional block diagram of a field programmable gate array suitable for use with the ultrasound system of FIG. 2;

FIG. 5 illustrates a translation of a physical to a virtual position of a transducer element;

FIG. 6A is a block diagram of a dynamic receiver focussing module suitable for incorporating in an embodiment;

FIG. 6B is a block diagram of a processor slice suitable for incorporating in the dynamic receiver focussing module of FIG. 6A;

FIG. 7 illustrates a interpolation process suitable for use with an embodiment;

FIG. 8 is a flow diagram of a method of operating an ultrasound probe to obtain an ultrasound image of a patient's bladder according to an embodiment;

FIG. 9 is a flow diagram of a method of detecting the presence of a pubic bone in an image frame according to an embodiment;

FIG. 10 is an example image frame displaying a pubic bone in a right most portion;

FIG. 11 is a distribution of normalised scanline sums for image frame of FIG. 10;

FIG. 12 is a flow diagram of a method of determining the location of the bladder anterior wall and bladder posterior according to an embodiment;

FIGS. 13A and 13B are simplified representations of discontinuities in a bladder wall outline.

FIG. 14 is flow diagram of a post-processing method for improving the accuracy of the detected bladder posterior and anterior wall locations according to an embodiment;

FIG. 15 is an example of a normally distributed scaling function suitable for use with embodiments;

FIG. 16A and FIG. 16B illustrate mirror artefact detected and resolution by a post processing algorithm according to an embodiment;

FIG. 17 illustrates an example of left sided mirror/gas artefact resolution according using a post-processing method to an embodiment;

FIG. 18 illustrates an example of right sided mirror/gas artefact resolution according using a post-processing method to an embodiment;

FIG. 19 is a flow diagram of a method of detecting bladder side walls according to an embodiment;

FIG. 20 is an ultrasound image showing including edge detector windows for a side wall.

FIG. 21 is a flow diagram of a method of calculating bladder volume from a plurality of processed image frames according to an embodiment;

FIG. 22 illustrates a diagrammatic representation of an image frame pair;

FIG. 23 illustrates a diagrammatic representation of the bladder outline included in each of the image frames of the image frame pair shown in FIG. 22;

FIG. 24 illustrates examples of different lateral translations of an ultrasound probe in use; and

FIG. 25 shows a construction of a geometry of the intersection of two planes which may be applied by embodiments to obtain a formulation of “rocking motion” with an arbitrary amount of lateral translation.

### DESCRIPTION OF EXEMPLARY EMBODIMENTS

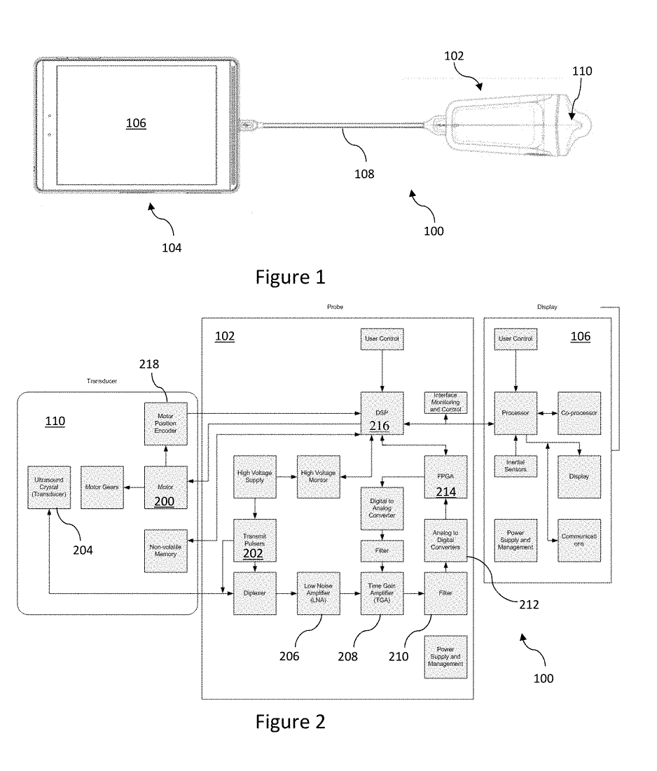

Referring to FIGS. 1 and 2, there is illustrated an ultrasonic imaging system 100 according to an embodiment of the invention. As shown, the system 100 includes a hand held ultrasound probe unit 102, a display and processing unit (DPU) 104 having a display screen 106 and, a cable 108 connecting the probe unit to the DPU 104.

In the illustrated embodiment, the probe unit 102 includes an ultrasonic transducer 110 containing at least one transducer element 204 (ref. FIG. 2) mechanically coupled to a motor 200 (ref. FIG. 2) and adapted to transmit pulsed ultrasonic signals into a medium to be imaged and to receive returned echoes from the medium. The ultrasonic transducer 204 preferably includes eight transducer elements (not shown) arranged in an annular array. However, it will be appreciated that a different number of transducer elements 204 and a different array arrangement may be suitable.

FIG. 2 shows a block diagram for an embodiment of the system 100. As shown, the system 100 includes a transmit pulser 202 which generates a short electrical pulse to create an oscillation in transducer elements 204 so that the transducer elements 204 generate an ultrasonic pressure pulse for transmission into the medium to be imaged.

The transducer elements 204 receive reflected ultrasonic pressure pulses and convert received pressure pulses into electrical signals, which are amplified by low noise amplifiers 206 provide amplified signals to a time gain amplifier 208. Bandpass or low pass filters 210 filter the resultant signals to provide a filtered analog signal. The filtered analog signals are then converted into digital signals via analog to digital converters 212.

Field programmable gate array (FPGA) 214 receives the digital signals in a low voltage serial format and deserialises the low voltage serial format signal, which are preferentially delayed to provide receive focussing (which will be explained further below). FPGA 214 then buffers and communicates an output signal to digital signal processing (DSP) unit 216 for processing. In the present case, the process of receiving reflected pulses and transferring to the digital signal processing unit (DSP) is defined as acquiring a scanline.

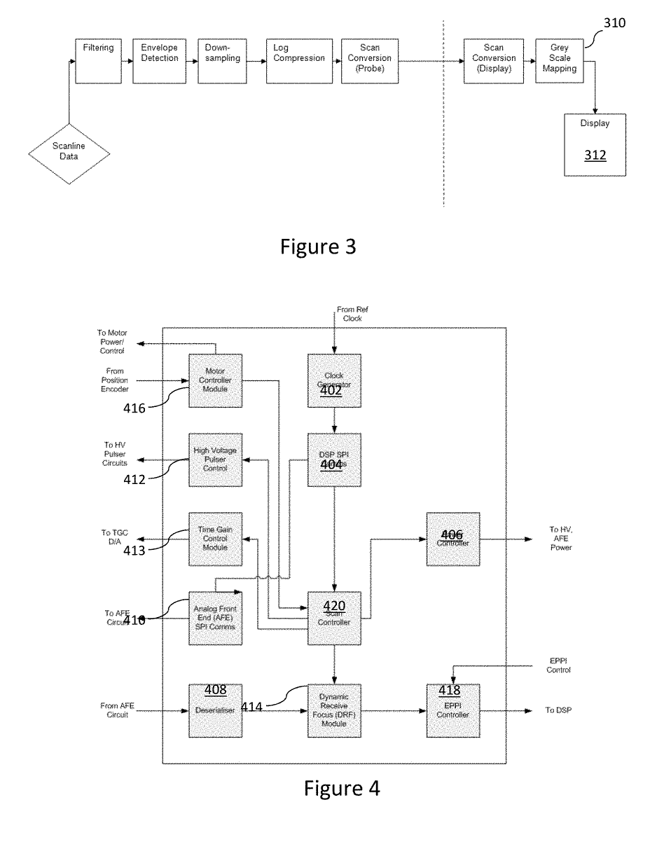

A functional block diagram of the DSP 216 processes is shown in FIG. 3. As shown, the digital signal processing device 216 processes each individually acquired scanline by applying a digital filter 300 to raw scanline data, detecting 302 an envelope of the scanline data, downsampling 304 the enveloped data, compressing 306 the raw input data (which is preferably 12 bits) into a low number of bits, and storing 308 the scanline for later scan conversion.

At the completion of a scanline transmit, acquisition, and processing process, the FPGA 216 will await the appropriate time to initiate transmission of the next pulse and repeat the process. In the illustrated embodiment, the time a pulse is transmitted is thus controlled by the FPGA 214.

Referring to FIG. 4, there is shown a block diagram of functional modules of the FPGA 214. As shown, the functional modules include a clock generator 402, DSPSPI Comms module 404, power control module 406, deserialiser 408, AFESPI comms module 410, HV pulser interface module 412, TGC interface module 413, Dynamic Receive Focus (DRF) module 414, motor controller module 416, EPPI controller module 418 and scan controller 420.

The DSPSPI Comms module 404 contains configuration memory setup by the DSP 216 prior to scanning. The configuration memory includes a scanline firing table, which is a table containing an encoder count for each scanline acquisition. For example, if 128 scanlines are required to generate a sector image, the scanline table contains 128 entries having the encoder position for each scanline. During scanning, the FPGA 214 receives a motor position encoder input from motor position controller 218 (ref. FIG. 2), converts the encoder input to a count, and compares the count to the DSPSPI Comms module scanline table. When the count matches a scanline firing position the FPGA 214 initiates a transmit pulse to acquire a scanline.

Returning now briefly to FIG. 3, having captured a set of scanline acquisitions covering the image area, the set of captured scanlines are packaged and communicated to the display processing unit 106 for scan conversion 308, grey scale mapping 310, image processing and display 312. Although not illustrated, part of the scan conversion 308 may be performed on the probe unit 102 in order to reduce the amount of data to send to the display processing unit 106, and therefore reduce the data transfer bandwidth required between the ultrasound probe and display processing unit 106.

Returning again to FIG. 4, FPGA 214 also includes a dynamic receive focus (DRF) module 414. The function of the DRF module 414 is to process, in this example, eight channels (that is, one channel per transducer element 204) of acquired data by selectively delaying and adding each channel together to provide constructive interference between channels, thereby increasing the system signal to noise ratio and improving system lateral resolution.

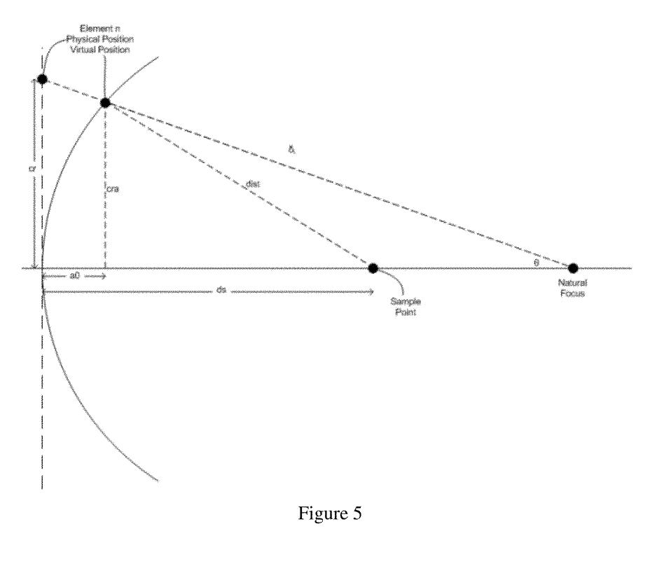

The conceptual requirement for dynamic receive focussing is illustrated in FIG. 5. As described above, the transducer elements 204 are arranged in an annular array format. A curve on the annular array elements 204, or other factors, may cause a focus to the transducer system which is used for calculating delays for each channel. To enable each element to be constructively added, the relative delay from a sample point to each element 204 in the array must be known or calculated.

The relative delay is directly related to the relative distance and focus. Referring to FIG. 5, by applying dynamic receive focus the physical position of the transducer elements 204 is adjusted by the focus of the system 100, resulting in a virtual position for each element 204. The distance from a sample point to the virtual position of an element is referred to as dist. Dist can be calculated using the right angle triangle bounded by dist, centre radius adjusted (cra), and distance from the sample point to be focussed to the centre element of the array (ds) less the annular offset (a0) of the element. That is:

dist={square root over (ds−ao)²+cra²)}

Both ao and cra can be calculated from the geometry of the annular array as follows:

$$θ = atan ( \frac{cr}{{δ}_{L}} )$$

Where cr or centre radius is equal to the physical distance from the centre element to the element for which the delay is being calculated, and δL is the distance from the natural focus of the transducer system to the physical element position for which the delay is being calculated. Therefore:

$$θ = atan ( \frac{cr}{{δ}_{L}} )$$

$$and$$

$$sin θ = \frac{cra}{{δ}_{L}}$$

### Therefore:

$$cra = {δ}_{L} {sin}^{f ( )} ( atan ( \frac{cr}{{δ}_{L}} ) )$$

### Also:

$$cos θ = \frac{{δ}_{L} - ao}{{δ}_{L}}$$

### Therefore:

$$ao = {δ}_{L} - {δ}_{L} cos ( atan ( \frac{cr}{{δ}_{L}} ) )$$

For a predefined sample frequency, a known geometry of the annular array, and a predefined interpolation oversampling factor, relative oversampled sample delays from a central transducer element to each other transducer element can be determined for every sample point.

The total memory required for an eight channel annular array would be the sample length (for example, 4096 samples in the preferred embodiment) times the number of element less one times the number of bytes to store each sample delay (two). This requires 56 kbytes of memory. The amount of memory can be reduced by taking advantage of the fact that the further the sample point is from the annular array the less the sample delays change. By formatting the delay memory as eight bits of course delay memory, two buts of fine delay memory, and six bits of repeat count, the required memory to store the delay parameters can be reduced by a factor of eight.

### Dynamic Receive Focusing

A method for dynamic receive focusing according to an embodiment involves receiving each scanline for the eight transducer elements 204 of the ultrasonic transducer 110, oversampling and interpolating the scanlines to enable finer resolution, delaying the scanlines by a required offset from the centre element, and adding the scanlines together. Since the delays change for every sample point, this operation is required to be performed for every sample point.

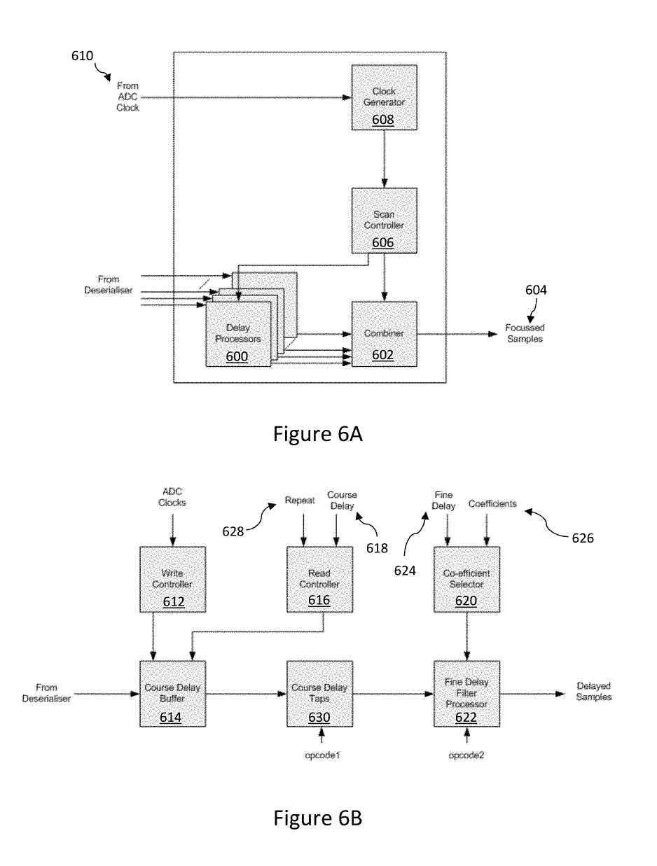

Another approach for implementing dynamic receive focussing (DRF) using reduced resources and lower power consumption is illustrated in FIG. 6A. FIG. 6A shows an overall structure of an exemplary DRF module 414. In the illustrated approach, input samples (ADC samples) from the deserialiser (ref. FIG. 4, item 408) are input into a respective processor slice 600 of the DRF module 414. A processor slice 600 is thus required for each transducer element 204 in the transducer array (preferably eight).

Each processor slice 600 delays a respective sample by a suitable delay to provide, as an output, delayed samples. A suitable delay may be 1/24^{th} or 1/48^{th} of a sample period, and up to 128 samples for a 20 mm transducer with 8 channels operating at a sample rate of 20 MHz. Combiner 602 then adds the delayed samples from each processor slice 600 to provide a final focussed output sample 604.

Scan controller 606 provides sequencing required to perform all of the DRF operations at the correct time, with the controller timing reference generated by the clock gen 608 module and directly related to the ADC clock (sampling clock) 610.

An example processor slice 600 configuration is shown in FIG. 6B. As shown, the processor slice 600 includes a write controller 612, a coarse delay buffer 614, and a read controller 616. Write controller 612 receives input samples and writes them into the coarse delay buffer 614. The coarse delay buffer 614 needs to be of sufficient length to allow for a maximum differential sample delay between a centre transducer element and the outside transducer element 204 of the transducer array. In an embodiment including eight transducer elements 204 and a 20 mm outside transducer diameter, a course delay buffer of 128 samples is required.

The course delay buffer 614 may be prefilled for each scanline to almost 128 samples. The read controller 616 then accesses prestored delay parameter data (prestored in the DSPSPI Comms module 404, ref. FIG. 4) to sequence the reading of the correct samples out of the buffer. Sufficient samples are pre-read out of the course delay buffer 614 and written to the fine delay buffer for use in the delay filters.

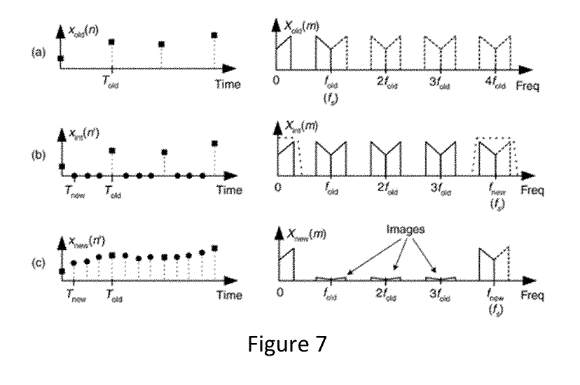

As explained above, a method for dynamic receive focusing according to an embodiment also involves oversampling and interpolating the scanlines to enable finer resolution. With reference now to FIG. 7, oversampling and interpolating each scanline requires a signal processor operating so that samples (T_{old}, ref. FIG. 7(a)) are zero padded (T_{new}, ref. FIG. 7(b)) to create a sequence at a new sample frequency (f_{new}) with “zeros” written to the new intermediate samples. The new sequence is then low pass filtered to generate the interpolated sequence (ref. FIG. 7(c)).

In an embodiment, a 24-tap FIR filter is used to filter the samples output from the course delay buffer 614 (which are input to the fine delay buffer), although a longer FIR filter could be used. If implementing a 24-tap FIR filter for every scanline imposed prohibitive processing power, a polyphase filter may be used and make use of fact that no zero padding is required for a polyphase implementation of an upsampler, and the output from each filter bank is selected according to the required delay. Therefore, only 1/upsamplerate×total taps is required to be calculated for each sample point. Compared to a conventional FIR upsampling filter, the computation required for the preferred oversampling rate of 4 and a 24-tap filter would be reduced by a factor of 16.

An implementation of a polyphase filter is depicted in the processing slice 600 shown in FIG. 6B.

For a 24-tap filter and 4× oversampling rate, a total of six samples are stored in the delay tap buffer. The six samples stored are defined by the course delay parameters 618 and read into the delay taps buffer from the course delay buffer 614 by the read controller 616. Course 618 and fine 624 delay parameters are received for each sample. The fine resolution delay parameters (two bits) 624 are used by the coefficient selector 620 to select the coefficients 626 for the relevant polyphase filter bank. The fine filter processor 622 uses the buffered delay taps 630 and selected coefficients to calculate only the relevant polyphase filter bank which generates the required delay.

The read controller 616 sequences the reading of the relevant parameters from memory. A repeat count 628 is tracked so where a delay value is repeated over multiple samples, new delay parameter data is not requested. When the repeat count 628 expires, new delay parameter data is read, and the course delay buffer output 614 is updated and the delay taps refilled 630.

The above describe method of dynamic receive focussing may also allow apodisation to be provided for the system 100. Apodisation parameters may be pre-stored and applied to every output sample by the filter processor. Any type of apodisation can be prestored and applied.

The above described DRF method may optimise memory usage by making use of the fact delay values change more slowly the further the sample is from the transducer, and therefore delay values repeat. The method may also optimises power consumption and reduce processes power by using a polyphase filter to implement delays by performing the upsampling and filtering for only the relevant delay.

### Bladder Scanning

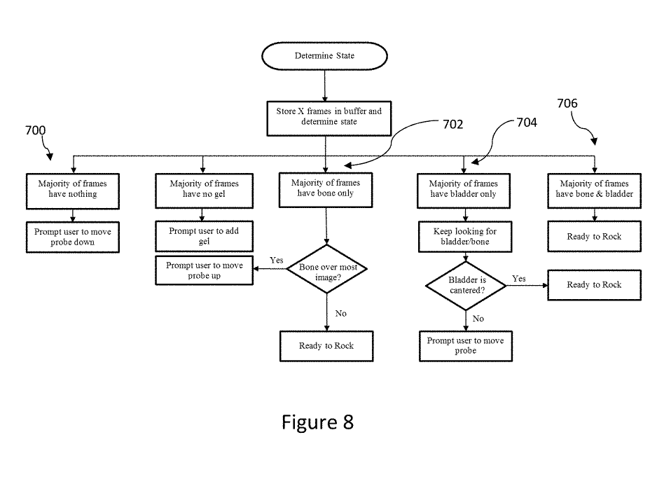

With reference now to FIG. 8, to obtain an ultrasound scan of a patient's bladder, the transducer 110 (ref. FIG. 1) is placed over the patient's abdomen and a plurality of ultrasound scans are captured in the form of a plurality image frames. The plurality of image frames are then processed to determine the positional state of the ultrasound probe relative to either an organ required to be scanned (in this example, the bladder) or a different anatomical structure of the patient having a known positional relationship with the organ and guide the user on what action to take so as to locate the bladder in the field of view of the ultrasound probe. In the below description, the different anatomical structure is the pubic bone, although it is to be appreciated that other anatomical structures may be used.

In general terms, a system 100 according to an embodiment applies the following criteria to determine the positional state of the probe unit 102 (ref. FIG. 1) and guide the user on what action to take so as to locate the bladder in the field of view of the ultrasound probe:
If the system 100 detects that a majority of image frames have no bladder or bone detected in the image frames (that is, branch 700), then the system prompt the user to move the probe unit 102 down towards the feet of the patient to look for the pubic bone which is an established landmark for locating the urinary bladder; or
If the system 100 detects that a majority of image frames have pubic bone only (that is, branch 702) where the pubic bone is over the majority of the image, the system prompts the user to move the probe unit 102 towards the patient's head as the probe unit 102 is placed too low over the pubic bone; or
If the system 100 detects that a majority of frames have bladder only (that is, branch 704), a centroid of the bladder is calculated and the user is prompted to move the probe to centre the bladder on a scan window if it is detected on the peripheral of the scan window. Once the bladder is adequately centred, the user is prompted to rock the probe unit 102 to capture the frames that will be segmented and used to calculate the bladder volume; or
If the processing detects that a majority of frames have pubic bone and bladder detected (that is, branch 706), then the location of the bone and the centroid of the bladder are used to prompt the user to adequately position the probe and rock it.

Although not shown in FIG. 7, if the system 100 detects that the pubic bone is located on the periphery of the scan window then the ultrasound probe is placed in the optimum position to view the patient's bladder and therefore the user is prompted to rock the probe to capture the image frames for segmentation and processing to calculate the bladder volume, as will be explained below.

### Pubic Bone Detection

As outlined above, processing of the image frames to determine the positional state of the probe unit 102 and guide the user on what action to take so as to locate the bladder in the field of view of the ultrasound probe unit 102 may involve detecting the presence of an anatomical feature, which in this case is the pubic bone, in the image frames. According to some embodiments, the location of the pubic bone is determined using a pubic bone detection algorithm. Once so determined, the location of the pubic bone may be used by a bladder segmentation algorithm to determine the start of the bladder. In embodiments, the pubic bone thus may be used a landmark to guide the user on accurate probe unit 102 placement so a majority of the bladder falls within the transducers' scan window (that is, the field of view of the probe).

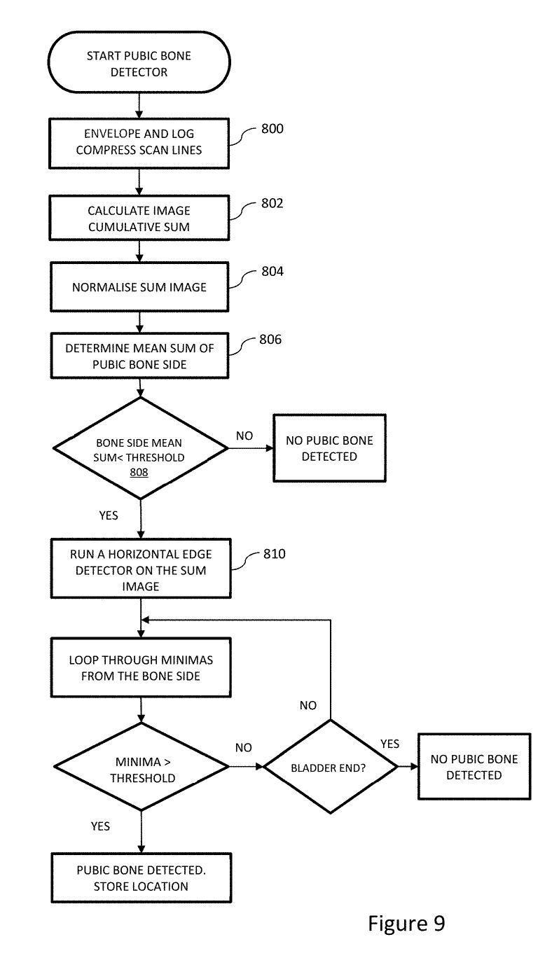

Turning now to FIG. 9, one embodiment of a pubic bone detection algorithm operates as follows.

First, raw RF echoes for each of the plurality of scanlines for a single scan window are filtered, enveloped, and log compressed 800 in a conventional manner to produce a conventional image frame for an ultrasound image. Notably, at this stage no scan conversion is performed on the processed scanlines as tracking and processing each individual scanline improves the operation of the pubic bone detection.

The greyscale values of each individual scanline are then summed 802 from a predefined dead zone. In the present case, the summation begins from around 2.5 cm (dead zone) as the first 2.5 cm typically contains image features resulting from reverberations, skin, adipose tissue. These image features are all echoic and occur at a shallower depth to the pubic bone. Therefore it is intended to start the summing at a greater depth to where a bone is located. The dead zone minimises the effect of the strong shallow reverberations which may otherwise hinder the detection algorithm. The scanline sums are then normalised at step 804 to reduce the effect of variations in image brightness and patient anatomy on the pubic bone detection algorithm. In an embodiment, normalisation involves:

$$Sn - \frac{s - min}{max - min}$$

### Where:

Sn is the normalised sum pixel;
S is the original sum pixel;
max is the maximum sum pixel value in the image; and
min is the minimum sum pixel value in the image.

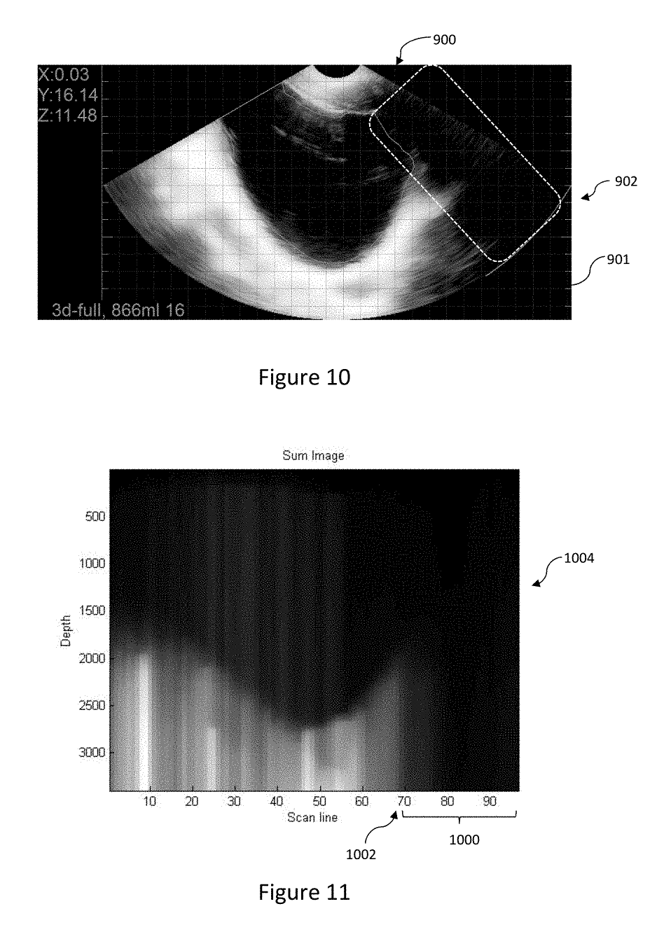

In general terms, the pubic bone detection algorithm recognises that scanlines passing through the pubic bone will have significantly lower sum values due to the blocking nature of bone to ultrasound signals. The pubic bone detection algorithm also assumes that the ultrasound probe 102 is orientated correctly. In this respect, an ultrasound probe may include a probe orientation marker which indicates the orientation of the ultrasound probe 102. In such a case, the orientation marker would be directed towards the patient's head. As shown in FIG. 10, in such an orientation, when the ultrasound probe 102 is located on the abdomen of the patient, the pubic bone is generally located on the right of the scan window 900 of the image frame 901. In the present case, the region of the scan window 900 including the pubic bone artefact is the region identified with a dashed rectangle.

Continuing now with a description of the operation of the pubic bone detection algorithm, and returning now to FIG. 9, the mean of the normalised sum of the greyscale value of the rightmost number of scanlines is calculated is then calculated at step 806. The number of scanlines used depends on the size of the detected bladder within the frame. The smaller the detected bladder, the more scanlines are used to ensure that the pubic bone is visible within a significant portion of the frame. A suitable range for the number of adjacent scanlines used to calculate the mean of the sum is 5% to 25% of the complete set of scanlines comprising the image frame. For example, in an embodiment comprising image frames having 128 scanlines, a suitable range for the number of scanlines used to calculate the mean of the sum is 10 to 30 lines. At step 808, in the event that the calculated mean is below a predefined threshold the image frame 901 is flagged as a potential bone frame. In an embodiment, the threshold is between 20% to 30% of the normalised image scanline sum range, with the actual threshold depending on the detected bladder size. With reference to FIG. 10, which depicts a distribution of the normalised scanline sums for image frame 901, the normalised sums in region 1000 is less than the predefined threshold and hence, in this example, the associated frame 901 (ref. FIG. 10) would be flagged as a potential bone frame.

Continuing again with reference to FIG. 9, having established that a frame is a potential bone frame, an edge detector filter is applied at step 810 horizontally along the normalised sum to determine the location where the bone shadow ends and thus where the bladder starts. The edge detector filter may include, for example, a differential spatial filter configured to calculate the edges across the scanline sums such that a first positive peak above a predefined threshold is set as the scanline where the bone shadow ends. In the present case, the first positive peak above a predefined threshold is set as the scanline where the bone shadow ends starting from the right of the scan window 900 (ref. FIG. 10). In the example shown in FIG. 11, the scanline having the first positive peak above a predefined threshold is shown as scanline 1002.

### Determining the Location of Bladder Anterior and Posterior Walls

In an embodiment, the individual scanlines are processed to determine the location of the bladder anterior wall and bladder posterior wall. In the present case, the determination of the location of the bladder anterior wall and bladder posterior wall involves processing the initial scanlines located after the end of the pubic bone shadow described above. In embodiments, if processing power or data storage are of concern, then processing the initial scanlines may involve processing enveloped and down sampled RF data.

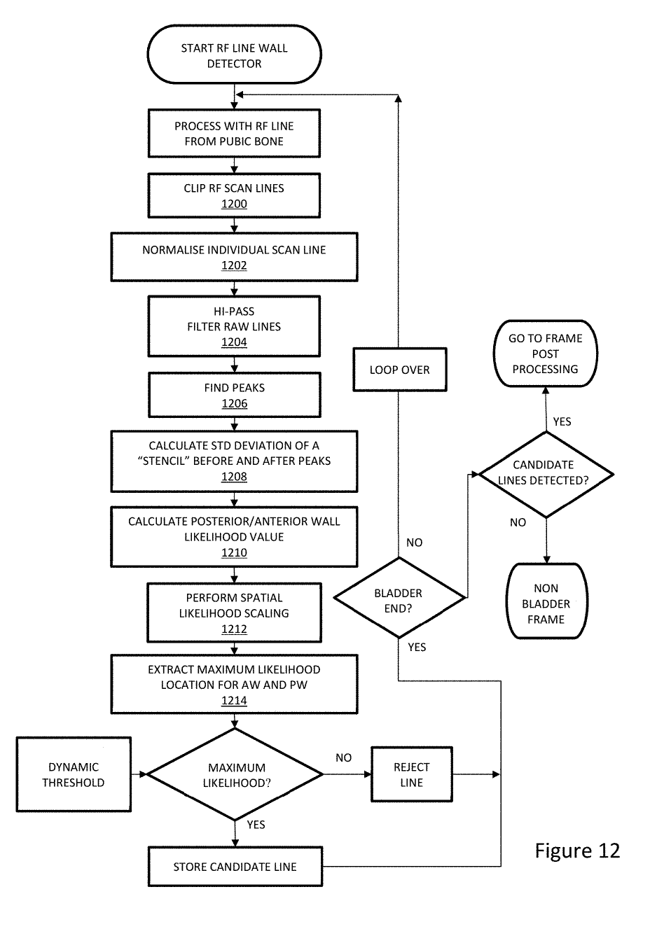

An approach for determining the location of the bladder anterior wall and bladder posterior wall by processing the initial individual scanlines located after (that is, to the left of) the end of the pubic bone shadow will now be described with reference to FIG. 12.

As shown in FIG. 12, each scanline is “clipped” 1200 to reduce the effect of excessively bright refractions which may reduce the performance of the algorithm. In an embodiment, clipping 1200 involves placing an upper limit on the maximum RF data value in the scanline. In an embodiment, the upper limit is set as 3600. Excessively bright reflections usually occur at tissue interfaces beyond the bladder posterior wall and have the potential to wrongly result in a large posterior wall likelihood value. Following clipping 1200, the scanlines are then individually normalised 1202 to decrease the algorithm's dependence on gain or differences in attenuation due to the patient's body fat composition among many other sources of image variations. The scanlines are normalised using the following formula:

$$Sn = \frac{s - min}{max - min}$$

### Where:

Sn is the normalised sample;
max is the maximum sample value within the scanline; and
min is the minimum sample value within the scanline.

A simple High Pass IIR filter is then applied 1204 to the scanline to remove any DC offset. One suitable filter is a 4th order butterworth filter with a cutoff frequency of 1 MHz. All peaks above a predefined threshold are detected and stored 1206 for the scanlines. In this respect, it has been found that a minimum peak value in the RF signal before the anterior wall and posterior wall is about 0.007 and 0.01 respectively, for a range of bladder images for different shapes and size. The wall likelihood is then calculated for all of these potential bladder locations. It is important to note that peaks for the posterior wall and peaks for the anterior wall will have different respective thresholds since the posterior wall usually has a larger reflection amplitude due to the posterior enhancement by from passing though the bladder.

The standard deviation (SD) of a set of RF signal samples bounded by a predefined window before and after each peak is then calculated 1208 and the likelihood of a peak laying on the bladder anterior wall or the bladder posterior wall is calculated 1210. If enveloped/down sampled RF data is used, a mean of the window may be used instead of the standard deviation as it is less computationally expensive and provides similar performance to the standard deviation.

The posterior wall likelihood PL is defined as:

$$PL = \frac{{SDP}^{0.5}}{{SDA}^{2}}$$

where SDA is the standard deviation of a predefined window anterior to (that is, before) the peak, and SDP is the standard deviation of a predefined window posterior to (that is, after) the peak. The likelihood function has a correlation with the bladder posterior wall because the bladder is always anechoic to some extent followed by a highly echoic region posterior to the bladder due to the very low attenuation of urine. The predefined window sizes are relatively large since we are not interested in detecting edges caused by speckle or reverberation artefacts. In the present case, a preferred posterior window size is about 1.2 cm and a selected chosen anterior window size is about 0.8 cm.

The anterior wall likelihood AL is defined as:

$$AL = \frac{SDA}{{SDP}^{ 2}}$$

The anterior wall likelihood function (AL) correlates with the bladder anterior wall because the urinary bladder is highly anechoic and is preceded by echoic fatty tissue.

The calculated posterior and anterior likelihood values are then spatially scaled 1212 by multiplying the individual likelihoods by a normal distribution function centred on the previous scanline's calculated bladder wall location and a standard deviation describing the normal deviation of the posterior wall (PW) and anterior wall (AW) seen in human bladders. This spatial scaling in effect is an anomaly detection function that detects and reduces outliers in the bladder wall. The spatially scaled likelihood value (L_s) can be expressed as:

Lₛ=SL

Where S is the scaling function and L is the original likelihood, that is, either the anterior likelihood (AL) or the posterior likelihood (PL).

$$S = {e}^{- \frac{0.5 {D}^{2}}{{STD}^{ 2}}}$$

Where D is the deviation in bladder wall location between the previous scanline and the current scanline likelihood location and STD is a predefined standard deviation of the bladder wall chosen from examining a large number of human bladders of many shapes and sizes. Care needs to be taken to ensure that standard deviation is sufficiently large enough to reduce the risk of wrongly rejecting scanlines for irregularly shaped bladders. It is important to note that the posterior wall has a higher standard deviation constant compared to the anterior wall as it typically has a higher variance.

The location of the candidate posterior wall (PW) and anterior wall (AW) is then stored for post processing to evaluate and improve the accuracy of the detected bladder wall locations.

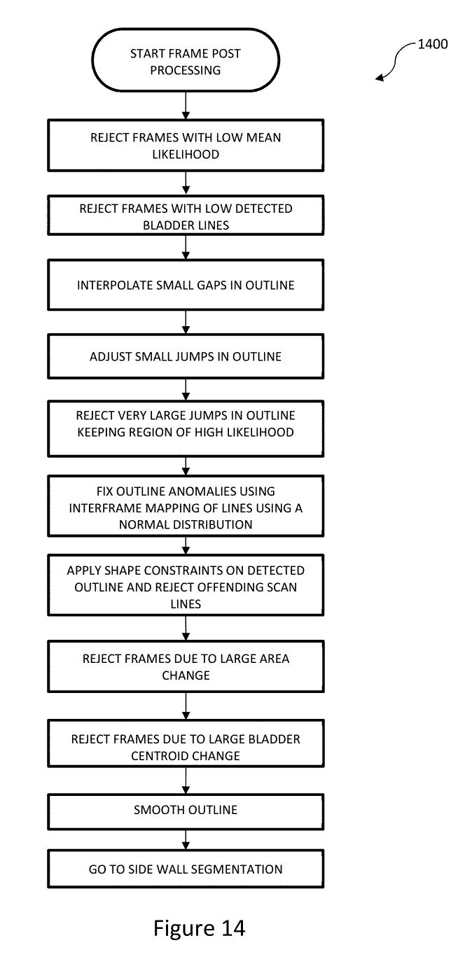

A flow diagram of an embodiment of a post-processing process 1400 for improving the accuracy of the detected bladder posterior and anterior wall locations is shown in FIG. 14. As shown, during post-processing, image frames with low average likelihood values (PL) and a very small number of detected bladder lines are first rejected as these are typically not bladder frames. In this respect, in an embodiment, a minimum of 6 bladder scanlines are required if no pubic bone has been detected, and 4 if a pubic bone has been detected. The average likelihood for the frame should be above 3000 if no pubic bone is detected and 2500 if a pubic bone has been detected. The RF line detector algorithm works on individual scanlines which may result in the algorithm missing some scanlines due to a weakly defined bladder wall or common ultrasound image artefacts. In the present case, the RF line detector algorithm interpolates the bladder wall in the presence of small gaps in the bladder wall given the number of undetected lines is below 13, the mean likelihood of the undetected lines is not below 1000 and the presence of bladder wall on either side of the gap.

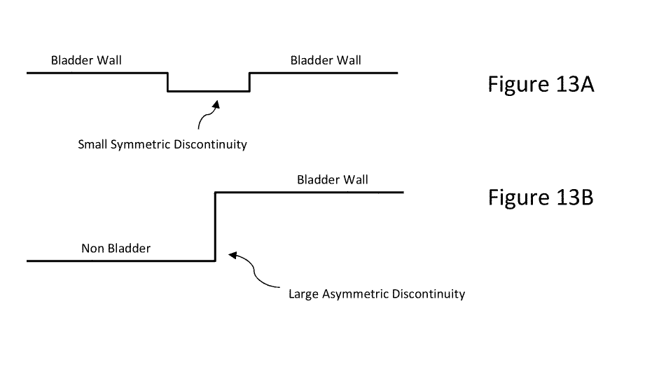

If the number of undetected lines is above the threshold or the mean likelihood of the undetected lines is below the threshold, then the proximity of the detected lines (that is, scanlines where a bladder wall has been detected by the RF line wall detector algorithm) to the pubic bone and the sum of detected lines' likelihood values may be used to determine which side of the “gap” the detected wall belongs to the bladder and which segment is potentially misclassified. As shown in FIG. 14, post-processing also scans for discontinuities in the form of “jumps” in the bladder wall. In this respect, if a discontinuity or “jump” is below a predefined threshold and is approximately symmetric, as illustrated in FIG. 13A, then in some embodiments the respective bladder wall is interpolated to remove the discontinuity. In the present case, a predefined threshold of 1.5 cm is used for the posterior wall and a predefined threshold of 0.75 cm is used for the anterior wall, although it will be appreciated that other values may be used. Preferably, linear interpolation is used to remove a discontinuity between the bladder wall location before the discontinuity and the bladder wall location after the discontinuity. If not removed, such discontinuities may be caused by reverberation artefacts on the bladder anterior wall and the bladder posterior wall.

On the other hand, if a discontinuity exceeds the predefined threshold and is approximately asymmetric, as shown in FIG. 13B, then it is considered that the system 100 has incorrectly classified another bladder like structure (uterus, cyst, or artefact) as the bladder wall. In this case, a segment of the bladder wall outline closer to the pubic bone, and with a larger total posterior wall likelihood, is classified as the bladder wall, while the segment of the bladder wall outline on the other side of the discontinuity is rejected. It is important to note that if the above described splines method is used then no interpolation or jump correction is required.

In embodiments, a large number of bladder image frames are captured (for example, 16 frames a second) as the user rocks the probe. The captured image frames are closely spaced (for example, less than 1° apart) and collectively image the patient's bladder. The availability of the closely related image frames improves the accuracy of bladder wall location and reduces anomalies in problematic noisy frames. An approach for reduces anomalies in problematic noisy frames will be described.

### Reducing Anomalies in Outlines

One method of reducing anomalies involves applying a normally distributed scaling function (S) which allows for normal inter-frame deviations in the outline of the bladder wall, or which “scales down” larger, abnormal inter-frame wall deviations.

The detected wall depth for a given scanline in a current image frame W₂ is described by:

W₂=W₁+W_{S}

### Where:

W₁ is the detected wall depth for the corresponding scanline in the neighbouring frame; and
Wₛ is the scaled difference.

In other words, if a bladder wall has been detected for scanline X then W2 is the depth where the bladder wall has been detected for this line, and W1 is the depth of the detected bladder wall location for the corresponding scanline in the previous frame.

The scaled difference Ws is the wall depth difference between neighbouring image frames multiplied by the maximum of a normally distributed scaling function (S) and a predefined constant (C).

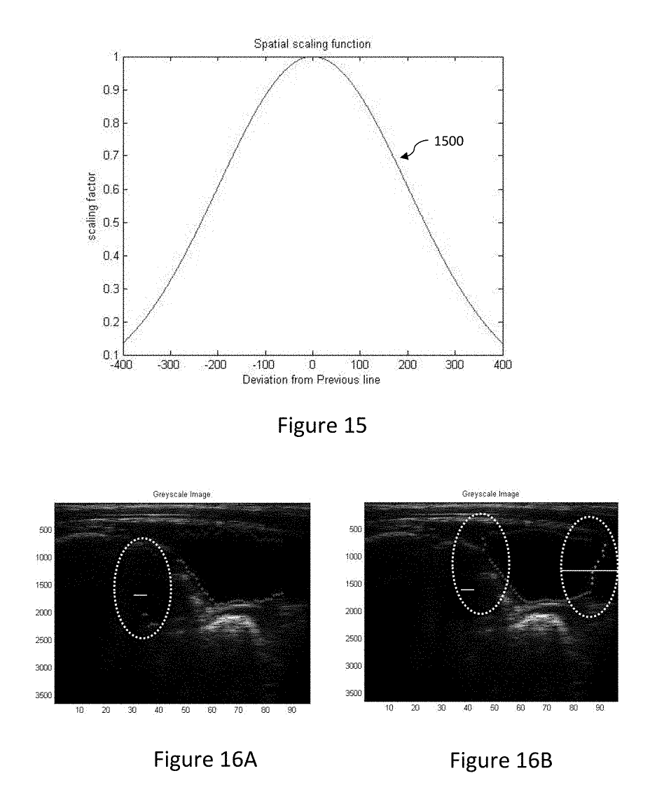

One example of a suitable normally distributed scaling function (S) 1500 is shown in FIG. 15. A normally distributed scaling function (S) 1500 of the type shown in FIG. 14 allows for normal deviations in the outline of the bladder wall compared to neighbouring image frames and passes them through as S will be ≈1. On the other hand, the scaling function 1500 will scale down large abnormal inter-frame wall deviations as S will be small. For example, with reference to the example scaling function 1500 shown in FIG. 15 it can be seen that S will have a value of 0.3 for a deviation of +/−300 samples.

A suitable scaling function (S) will preferably have a predefined bladder wall standard deviation (STD), inter-frame angle difference (Ang), and the inter-frame wall depth difference (W_{D}) as inputs as described in the below equation. A different standard deviation is used for the bladder anterior wall and the bladder posterior wall as the posterior wall typically has higher variance. The predefined constant C is chosen to prevent the algorithm from getting trapped.

W_{S}=W_{D}Max(S,C)

### Where:

$$S = {e}^{- \frac{0.5 {W}_{D}^{2}}{{( STD . Ang )}^{2}}}$$

It is noted that, in the above expression, having a large depth difference W_{D} or a large interframe angle Ang will result in S being ≈0. This will mean that the bladder location for scanline x in the current frame will be exactly the same as scanline x in the neighbouring frame. Also all subsequent frames will have large angle and depth differences as the last accepted depth for scanline x will be the last depth before the jump occurred. Accordingly, by selecting a suitable value for C, (eg. C=0.2) ensures that a minimum of 20% of the difference in bladder depth W_{D} is passed through.

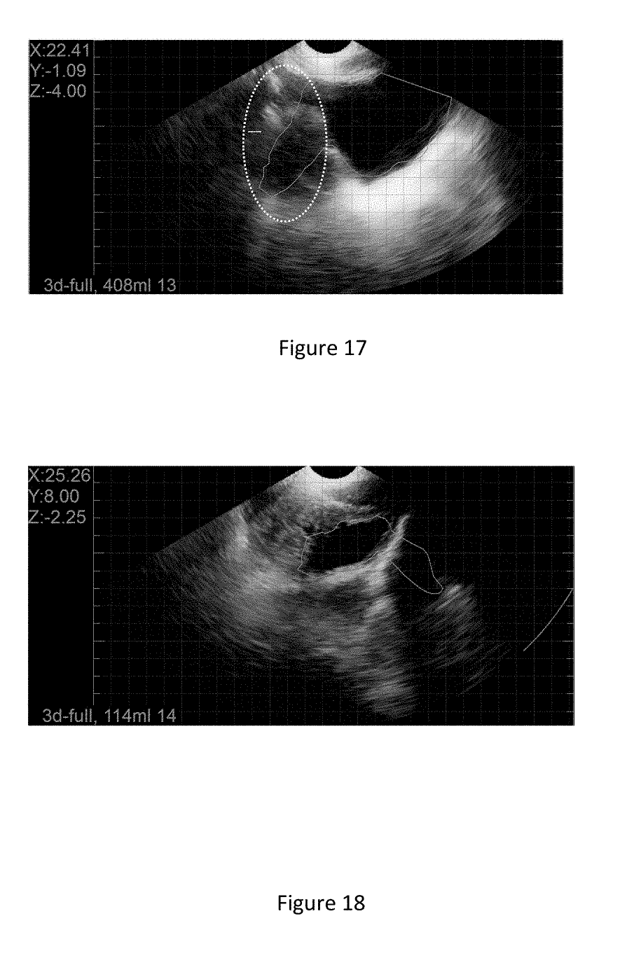

The bladder posterior wall naturally assumes a concave up shape profile. However, mirror/bone artefacts may cause the bladder posterior wall to assume a highly concave down shape, as seen in FIGS. 16A to 18. Preferably, in some embodiments of the present invention, post processing detects and adjusts anomalies in the detected bladder wall using a bladder posterior wall first derivative, in other words, the slope of the bladder posterior wall. In this respect, if the slope of the bladder posterior wall on the left side of the image (Gas/Mirror artefact side) is higher than a predefined threshold this may indicate that the bladder outline has been “pulled down”, such as by a mirror artefact or a gas artefact, as shown by region 1700 (ref. FIG. 16A and FIG. 17). Such lines are tagged as artefact lines and omitted from the bladder outline as shown in FIG. 1B. A similar process is repeated in the region 1800 illustrated on the right side of the image (ref. FIG. 18) with the result of this process (and the process applied to region 1700) illustrated in FIG. 16B. In one embodiment, a threshold of 1.5 mm/scanline slope is used.

Next, post-processing determines an inter-frame change in area of the bladder outline centroid and this information, together with the angle difference between neighbouring frames, is used to determine the validity of the image frame as the bladder shape and location are bounded. In the present case, this step is performed after the bladder anterior wall, posterior wall, and side walls have been determined. A bladder polygon is then formed using the four aforementioned walls and the area and centroid is calculated for each frame.

In the present case, if the area of the bladder polygon changes by more than 30% per degree and by more than 0.001 m² between frames then the image frame is rejected. Furthermore, in the present case, if the bladder centroid between adjacent frames moves my shifts by than 15 pixels per degree or a maximum of 50 pixels regardless of angle then the frame is also rejected. Methods according to embodiments can afford to drop frames because a large number of frames is captured in a typical scan (average is around 80 frame) which means that the bladder is oversampled and dropping up to half the frames has minimal impact on the volume calculation. Finally, the detected bladder walls are passed to a simple averaging filter to smooth the bladder outline.

### Determining the Location of Bladder Left and Right Side Walls

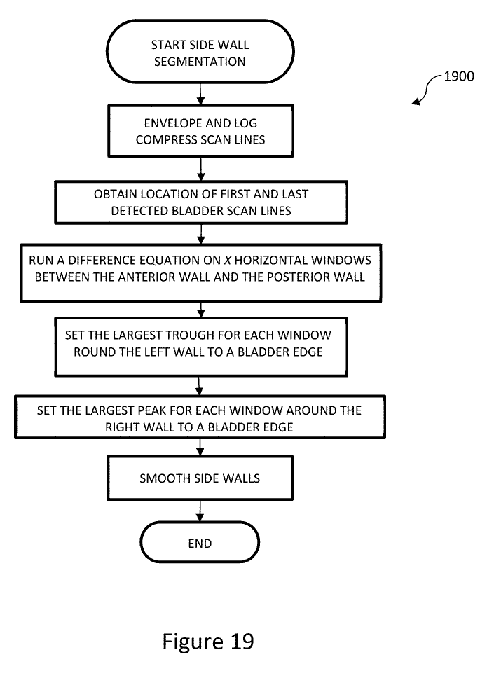

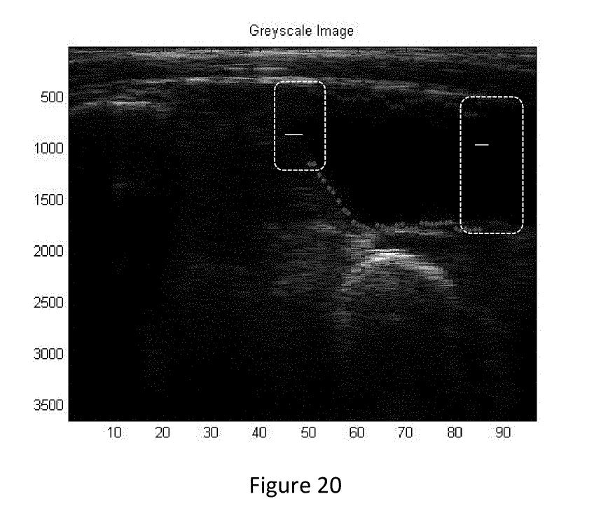

FIG. 19 is a flow diagram of a method 1900 of detecting the bladder side walls. As shown, in some embodiments, a conventional differential edge detector is used to detect the bladder's left and right side walls. Each side wall outline is typically very weak and prone to a large number of artefacts. Therefore, in embodiments, each edge detector is bound by a predefined window 2002, 2004 (ref. FIG. 20). In the present case, each predefined window 2002, 2004 is sized to include eight scanlines located towards the exterior of the bladder and three scanlines located towards the interior of the bladder. In other words, a total of eleven scanlines is used in each window 2002, 2004, but off centre as shown in FIG. 20.

In the present case the edge detector filter is a differential spatial filter used to calculate the edge value on a sample of horizontal lines between the bladder anterior wall and posterior wall.

E(r)=(1/I(r)^{0.5}){I(r+2Δr)+I(r+Δr)+I(r)−I(r−Δr)−I(r−2Δr)}

### Where:

E is the edge value; and I is the pixel grey level value.

For the right wall 2004, only the transition from dark regions to bright regions is significant since the edge detection algorithm starts from a point within the bladder area, and it is known that the bladder is the darkest region within the image, hence only positive edge value are used. On the other hand, for the left wall 2002, only the transition from bright regions to dark regions is significant since the edge detection algorithm starts from a point outside the bladder area to a point inside the bladder area, hence only negative edge value are used. An averaging filter is then used to smooth the side walls.

### Bladder Volume Determination

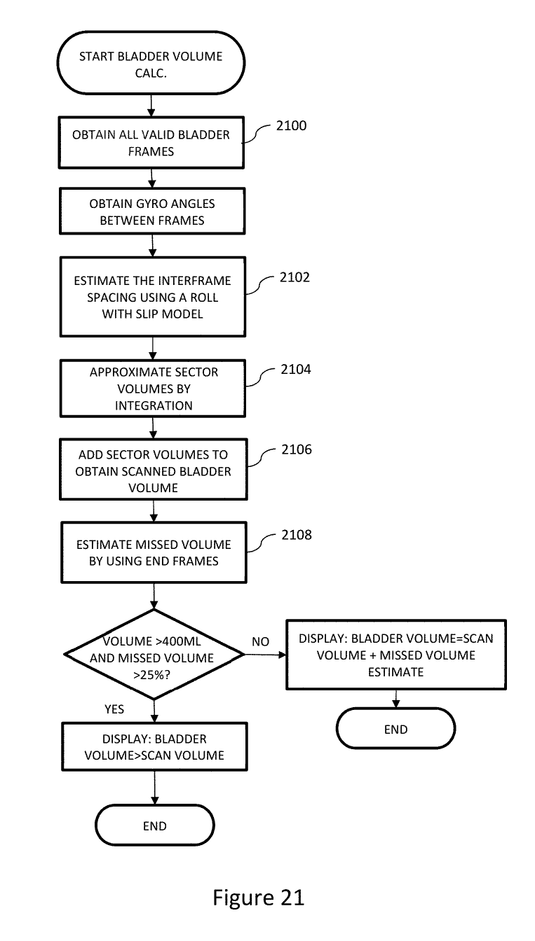

Now referring to FIG. 21, in embodiments, the volume of the bladder is calculated from the segmented bladder outlines and the angle between the respective frames associated with each outline, as acquired from the gyroscope. As shown in FIG. 21, one suitable approach of calculating bladder volume involves obtaining, at step 2100, all valid bladder frames (that is, each frame having an associated detected bladder outline), using the angle between said valid frames acquired from the gyroscope to determine, at step 2102, an inter-frame separation as a function of depth, and then integrating between frame pairs, at step 2104, to acquire a volume for the frame pair. The volumes determined for each frame is then added, at step 2106, to obtain the total scanned bladder volume

In some cases, where a bladder is not completely captured by the scan, a missed volume may be extrapolated, at step 2108, using the last detected “valid frame” and a model of the bladder. As shown in FIG. 21, the extrapolated volume should not exceed a predefined percentage of the scanned bladder volume to maintain an accurate bladder volume estimation.

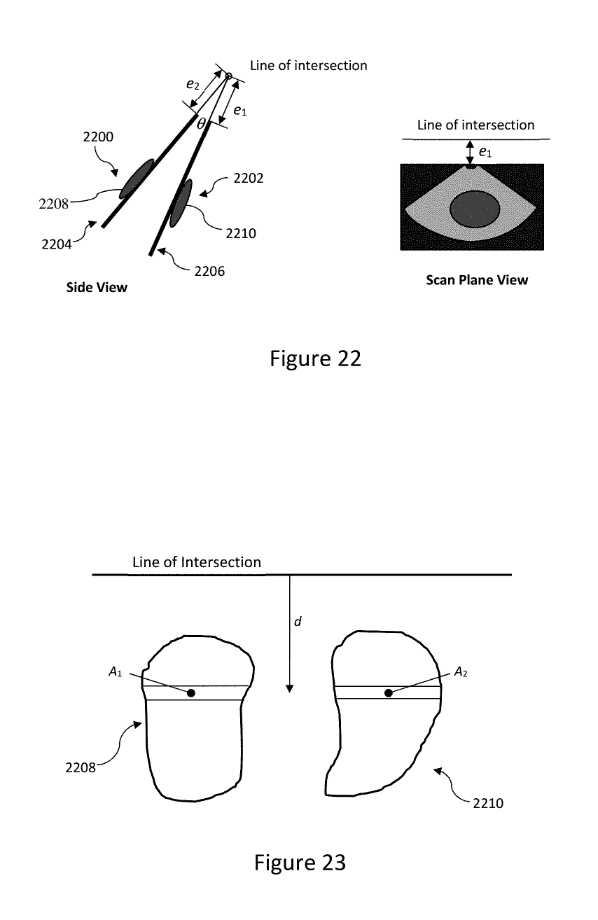

Referring now to FIG. 22, the total bladder volume is thus calculated by accumulating a sequence of volume “slices”, with each slice is bounded by two scan frames. FIG. 22, depicts two image frames images, namely a first image frame 2200 and a second image frame 2202, in a side view, and one of the images frames in the scan plane view. In the side view, lines 2204, 2206 represent the respective images frames and the “ovals” 2208, 2210 represent the respective position of the bladder outline within each scan image frame. Note that the planes of the image frames can be extended to meet at a line of intersection, that is, the point from which the transducer array transmits the ultrasound pulses into the medium. To calculate the volume of the “slice” requires the angle between the scan planes and the distances from the line of intersection to the top of each scan image, e1 and e2, which can be different.

To compute the slice volume from the bladder outline 2208, 2210 in each frame 2200, 2202 integration over the depth d is performed as shown in FIG. 23.

The volume contribution, W, from a slice a given slice depth is from,

$$W = \frac{( {A}_{1} + {A}_{2} )}{2} d θ$$

### Where:

A₁ and A₂ are the areas of the slices in outlines 2208, 2210 respectively at a common depth, d, in the image, measured from the line of intersection.

By applying this equation across the full depth of each image frame 2208, 2210 and summing the results, the total volume of the “slice” between the two image frames 2208, 2210 is obtained.

To obtain the total measured bladder volume, the volume of all the slices is summed. In some embodiments, an allowance for the volume at each end of the sweep beyond the last frame with a detected outline is incorporated. In this respect, the volume allowance at each end of the sweep may be calculated as follows:
The area of each segmented frame is plotted against the angle of the frame.
A curve is fitted to the plotted data and extrapolated to zero area in each direction.
If the curve converges to zero area and the extrapolated volume is greater than 25% of the total volume, then an integration of the area under the curve is used to estimate the end volume.
If the curve does not converge to zero area, or the extrapolated volume is greater than 25% of the total volume, then the result is reported to the user as greater than the calculated figure.

### Compensating for Probe “Rolling” or “Slipping”

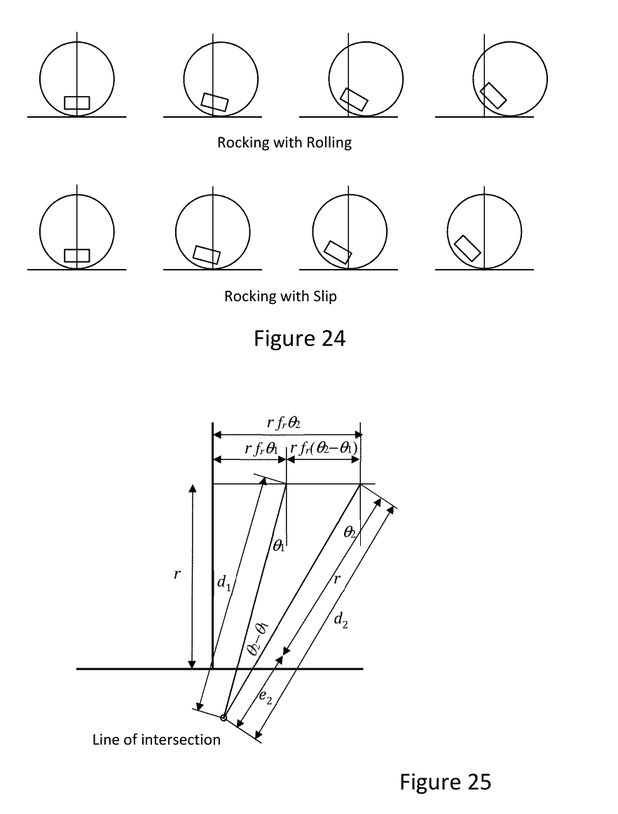

The volume calculation approach described above assumes the transducer was pivoted at a single point at the tip of the transducer. However, as the user rocks or pivots the probe, different amounts of lateral translation may be possible, as shown in FIG. 24. The circles in the diagram represent the side view of the probe as it is rock on its hemispherical head.

In the ‘Rocking with Rolling’ case, the probe rolls across the patient's skin like a tyre. In ‘Rocking with Slipping’ case the probe slips on the patient's skin, such that the centre of the probe head stays in the same position.

Turning now to FIG. 25, there is shown a construction of a geometry of the intersection of two planes which may be applied by embodiments to obtain a formulation of “rocking motion” with an arbitrary amount of lateral translation.

In FIG. 25:
r, is the radius of the transducer head;
θ₁ and θ₂ are the angles of the two scan planes; and
fr is the ‘roll factor’. fr equal to 1 corresponds to the ‘rock with roll’ case and fr equal to 0 corresponds to the ‘rock with slip’ case.

As discussed above, in embodiments calculating the volume between two slices involves calculating the distances d from the line of intersection to the top of each scan image, e1 and e2, which may be calculated as follows:

$${e}_{1} = r - \frac{{rf}_{r} ( {θ}_{2} - {θ}_{1} ) cos {θ}_{2}}{sin ( {θ}_{2} - {θ}_{1} )}$$

$${e}_{2} = r - \frac{{rf}_{r} ( {θ}_{2} - {θ}_{1} ) cos {θ}_{1}}{sin ( {θ}_{2} - {θ}_{1} )}$$

As describe previously, in embodiments, the probe unit 102 includes a three axis accelerometer. By subtracting the gravity vector and performing a double integral over time the position of the transducer can be tracked. The probe unit 102 periodically fires single or multi-cycle pulses within each scan frame, and performs Doppler analysis on the each of these pulses. A wide Doppler ‘gate’ is used at a shallow depth where little or no tissue movement is expected, which mean that any Doppler shift detected can be attributed to movement of the probe with respect to the tissue being measured. By using the angle returned by the inertial sensor system, the Doppler shift can be translated to a lateral motion, and then by performing a single integral can be translated to a distance or slip measurement.

The accelerometer and Doppler shift provide only a limited and relatively inaccurate measurement of the translation of the probe. However the translational motion of the probe is not independent of the rotation of the probe, which can be accurately measured. By using the general model of how the user translates the probe during rocking defined above and estimating the ‘roll factor’ parameter the accurate rotation measurement can be used to compensate for the inaccurate translation measurement. The accelerometer and Doppler shift measurements are combined and used to determine the ‘roll factor’. The final bladder measurement is determined according to one of the following three cases:
If the accelerometer and Doppler shift measurements are in agreement with each other and with the ‘roll factor’ model then the exact ‘roll factor’ is determined and the model is used to calculate the volume of the bladder from the measured slice angles.
If the accelerometer and Doppler shift measurements disagree with each other or with the ‘roll factor’ model then limiting ‘roll factor’ is determined such that the bladder volume will not be underestimated, the model is used to calculate the volume of the bladder from the measured slice angles and the result is reported to the user as greater than the calculated figure.
If the accelerometer and Doppler shift measurements indicate a gross departure from the proper rocking technique then the scan is terminated with an error and the user is prompted to repeat the scan.

### User Interface

FIG. 9 above detailed an approach for detecting the presence of the pubic bone in an image frame, which is preferably used to ensure that a volume measurement does not miss the bladder. In embodiments, a user interface with an instruction use model is provided to link the approach with user input. For example, embodiments may provide an instructional video which is activated by a user interface button, and which provides pre-process operating instructions for conducting a bladder volume determination, including, for example, positioning the patient, positioning the operator, application of ultrasound gel to the patient, positioning the probe on the patient, the method to grip the ultrasound probe and the method for rocking the ultrasound probe to obtain a three dimensional data set.

When the user activates a bladder scan measurement, which may involve, for example, pressing a “bladder scan start button” on the user interface, short step-by-step instructional videos and instruction are displayed, which are context sensitive. The videos may show, for example, a method of applying ultrasound gel to the patient and positioning the probe on the patient, movement of the probe up the patient's body if the position guidance algorithm requires it, movement of the probe down the patient's body if the position guidance algorithm requires it, and method for rocking the ultrasound probe to obtain a three dimensional data set. Each video may be accompanied by a short text description of the action required.

A position guidance algorithm according to an embodiment operates using the output from the pubic bone detection algorithm (FIG. 8) and bladder wall detection algorithm (FIG. 12). If the bladder wall detection algorithm has detected the bladder, and the bladder is only partially in the field of view, the position guidance algorithm causes the user interface to prompt the user to move the probe either towards the head (where the bladder is on the left of the image) or to the feet (where the bladder is to the right of the image). Where no bladder is detected, the pubic bone algorithm is used. Where no pubic bone is in the field of view, the position guidance algorithm causes the user interface to prompt the user to move the probe towards the patient's feet until either a bladder or a pubic bone is in the field of view. When the bladder wall detection algorithm has detected the bladder in the field of view, the position guidance algorithm causes the user interface to prompt the user to pivot or rock the transducer to fan through the bladder and acquire a 3D dataset. The position of the probe relative to the initial position is detected by inertial sensors in the probe, consisting of gyroscopes and accelerometers.

The provision of a user interface with prompts enables the ultrasound image to be hidden and the short step by step instructional videos displayed at a larger size. This may simplify the user experience even further for users who have not be trained to interpret the ultrasound image.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system 100. Skilled artisans may implement the described functionality in varying ways for each particular application, but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Further, it should be appreciated that modules and/or other appropriate means for performing the methods and techniques described herein can be downloaded and/or otherwise obtained by computing device. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a computing device can obtain the various methods upon coupling or providing the storage means to the device. Moreover, any other suitable technique for providing the methods and techniques described herein to a device can be utilized.

In one form the invention may comprise a computer program product for performing the method or operations presented herein. For example, such a computer program product may comprise a computer (or processor) readable medium having instructions stored (and/or encoded) thereon, the instructions being executable by one or more processors to perform the operations described herein. For certain aspects, the computer program product may include packaging material.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

1. A system for guiding a user to aim an ultrasound probe to capture one or more image frames of an organ of a patient, the system including:
an image processor for capturing one or more image frames of a field of view of the ultrasound probe;
detecting means for processing the one or more captured image frames to detect the presence of a predetermined anatomical feature of the patient having a known positional relationship with the organ; and
guiding means for directing the user to adjust the aim and/or position of the ultrasound probe so as to locate the organ within the field of view of the ultrasound probe based on the known positional relationship.

2. The system of claim 1 wherein the organ is the bladder and wherein the predetermined anatomical feature is the pubic bone of the patient.

3. The system of claim 2 wherein the detecting means includes a processor for processing a range of scanlines of a set of scanlines comprising an image frame to:
determine a summation of intensity values for each of the scanlines within the range;
determine a mean summation value for the range;
compare the mean summation value for each range to a predetermined bone detection threshold value;
identifying a frame as a candidate pubic bone frame depending on the comparison.

4. The system of claim 4 wherein the range of scanlines includes a plurality of adjacent scanlines of the respective set of scanlines such that the plurality of adjacent scanlines comprises 5% to 25% of the set of scanlines of the respective image frame.

5. The system of claim 5 wherein the bone detection threshold has a value depending on a normalised summation of intensity values for the respective set of scanlines of the respective image frame.

6. The system of claim 3 wherein the detection threshold is a value in the range of 20% to 30% of the normalised summation of intensity values for the respective set of scanlines.

7. A method of guiding a user to aim an ultrasound probe to capture one or more image frames of an organ of a patient, the method including:
capturing one or more image frames of a field of view of the ultrasound probe;
processing the one or more captured image frames to detect the presence of a predetermined anatomical feature of the patient having a known positional relationship with the organ; and
directing the user to adjust the aim and/or position of the ultrasound probe so as to locate the organ within the field of view of the ultrasound probe based on the known positional relationship.

8. The method of claim 7 wherein the organ is the bladder and wherein the predetermined anatomical feature is the pubic bone of the patient.

9. The method of claim 8 wherein processing the one or more captured image frames to detect the presence of the pubic bone includes processing a range of scanlines of a set of scanlines comprising an image frame to:
determine a summation of intensity values for each of the scanlines within the range;
determine a mean summation value for the range;
compare the mean summation value for each range to a predetermined bone detection threshold value;
identify a frame as a candidate pubic bone frame depending on the comparison.

10. The method of claim 9 wherein the range of scanlines includes a plurality of adjacent scanlines of the respective set of scanlines such that the plurality of adjacent scanlines comprises 5% to 25% of the set of scanlines of the respective image frame.

11. The method of claim 10 wherein the bone detection threshold has a value depending on a normalised summation of intensity values for the respective set of scanlines of the respective image frame.

12. The method of claim 11 wherein the detection threshold is a value in the range of 20% to 30% of the normalised summation of intensity values for the respective set of scanlines.

13. A system for determining the volume of urine in a patient's bladder including:
an ultrasound probe for capturing a plurality of ultrasound image frames;
a memory storing a set of program instructions;
one or more processors programmed with the set of program instructions for execution to cause the one or more processors to:
process the plurality of captured image frames to detect an estimated location of the patient's pubic bone;
use the estimated location of the pubic bone to estimate the location of the bladder;
provide output information guiding the user to adjust the placement of the ultrasound probe according to the estimated location of the bladder so as to obtain a plurality of image frames including the bladder; and
process the plurality of image frames of the bladder to determine the volume of urine in the bladder.

14. A method for determining the volume of urine in a patient's bladder including:
operating an ultrasound probe to capture a plurality of ultrasound image frames;
processing the plurality of captured image frames to detect an estimated location of the patient's pubic bone;
using the estimated location of the pubic bone to estimate the location of the bladder;
guiding the user to adjust the placement of the ultrasound probe according to the estimated location of the bladder so as to obtain a plurality of image frames including the bladder; and
processing the plurality of image frames of the bladder to determine the volume of urine in the bladder.

15. A method for determining the volume of urine in a patient's bladder including:
operating an ultrasound probe to capture a plurality of ultrasound image frames including an image of the bladder;
processing each of the plurality of captured image frames to:
determine the location of the bladder's anterior and posterior walls using an estimated location of the pubic bone;
process scanlines including features of the anterior and posterior wall to select a respective range of scanlines proximal to end points of the anterior and posterior wall;
apply an edge detection filter to each respective range to detect left and right side walls of the bladder based on intensity transitions across the extent of each respective range; and
determine a volume slice using the detected walls of the bladder;
determining the volume of urine in the bladder as the summation of each volume slice.
